(19) United States
(12) Patent Application Publication
(10) Pub. No.: US 2026/0108175 A1
(43) Pub. Date: Apr. 23, 2026

(54) IDENTIFYING ACTIVITIES USING SENSOR DATA

(71) Applicant: Meta Platforms Technologies, LLC, Menlo Park, CA (US)

(72) Inventors: Eric Andrew ROSEN, Providence, RI (US); Doruk SENKAL, Kirkland, WA (US)

(21) Appl. No.: 17/546,573

(22) Filed: Dec. 9, 2021

(51) Int. Cl. A61B 5/11; A61B 5/00; G06N 3/045; G06N 3/08

(52) U.S. Cl. CPC A61B 5/1118; A61B 5/6802; A61B 5/6813; G06N 3/045; G06N 3/08

(57) ABSTRACT

A method for identifying activities is provided. The method involves obtaining a training set, the training set comprising training samples, a training sample comprising: a sequence of sensor data obtained from sensors disposed on a user spanning a first time duration, and a label of an activity the user was engaged in during collection of the sequence of sensor data. The method involves training a two-stage neural network to generate an output indicating a corresponding label of the activity, wherein the two-stage neural network comprises: a low-level encoder configured to receive a subset of the sequence of sensor data spanning a second time duration as input and generate a low-level output; and a high-level encoder configured to receive low-level outputs generated by the low-level encoder and generate the output indicating the corresponding label of the activity.

### BACKGROUND

With the increasing use of wearable computers, such as smart watches, fitness trackers, smart clothing, virtual reality (VR) or augmented reality (AR) headsets, or the like, identifying user activities is of interest. For example, by identifying an activity a user is currently engaged in, various actions may be triggered, such as causing contextually relevant information (e.g., reminders, weather forecasts, etc.) to be presented. However, it may be difficult to identify activities based on sensor data, such as motion sensor data. For example, in some cases, identifying activities based on sensor data may require massive training sets, which are computationally expensive to utilize and difficult to obtain.

### SUMMARY

In some aspects, a method for identifying activities includes: obtaining a training set, wherein the training set comprises a plurality of training samples, a training sample of the plurality of training samples comprising: a sequence of sensor data obtained from one or more sensors disposed on a user spanning a first time duration, and a label of an activity the user was engaged in during collection of the sequence of sensor data; training a two-stage neural network to generate, for each training sample in the training set, an output indicating a corresponding label of the activity, wherein the two-stage neural network comprises: a low-level encoder configured to receive a subset of the sequence of sensor data spanning a second time duration as input and generate a low-level output, the second time duration shorter than the first time duration, and a high-level encoder configured to receive a plurality of low-level outputs generated by the low-level encoder on a plurality of subsets of the sequence of sensor data and generate the output indicating the corresponding label of the activity; and providing one or more parameters associated with a trained two-stage neural network to a user device, such that the user device uses the one or more parameters to identify activities based on sensor data.

In some examples, the second time duration is less than about two seconds.

In some examples, the first time duration is more than about 30 seconds.

In some examples, the sequence of sensor data comprises at least one of: accelerometer data, gyroscope data, pressure sensor data, magnetometer data, or ambient light senor data.

In some examples, the low-level encoder is a fully connected network, a recurrent neural network, a long short-term (LSTM) network, a gated recurrent unit (GRU) network, a one dimensional convolutional neural network (1-D CNN), or a temporal convolutional network (TCN).

In some examples, the high-level encoder is a fully connected network, a recurrent neural network, a long short-term (LSTM) network, a gated recurrent unit (GRU) network, a one dimensional convolutional neural network (1-D CNN), or a temporal convolutional network (TCN).

In some examples, training the two-stage neural network comprises: partitioning the sequence of sensor data into the plurality of subsets of the sequence of sensor data based on a plurality of time windows; and providing each of the plurality of subsets of the sequence of sensor data to the low-level encoder. In some examples, each time window of the plurality of time windows is the same. In some examples, at least two time windows of the plurality of time windows are at least partially overlapping or have different durations.

In some examples, training the two-stage network comprises: determining, for a training sample in the training set, an error associated with a predicted activity generated by the high-level encoder relative to the corresponding label of the activity; and updating weights for the low-level encoder and the high-level encoder based on the error.

In some examples, the subset of the sequence of sensor data is filtered by a filter prior to being provided to the low-level encoder, and wherein the filter comprises: a low-pass filter, a high-pass filter, a bandpass filter, or a notch filter.

In some examples, the method further includes, prior to providing the plurality of subsets of the sequence of sensor data to the low-level encoder: determining a linear combination of at least a portion of the plurality of subsets of the sequence of sensor data; and applying an arithmetic operation to the linear combination.

In some aspects, a method for identifying activities includes: obtaining a sequence of sensor data obtained from one or more sensors disposed on a user, the sequence of sensor data spanning a first time duration; partitioning the sequence of sensor data into a plurality of subsets of sensor data, each subset of sensor data spanning a time duration less than the first time duration; providing each subset of the plurality of subsets of sensor data to a low-level encoder, wherein the low-level encoder generates, for each subset of the plurality of subsets of sensor data, a low-level output such that a plurality of low-level outputs corresponding to the plurality of subsets of sensor data is generated by the low-level encoder; and providing the plurality of low-level outputs to a high-level encoder to generate a prediction of an activity the user was engaged in during collection of the sequence of sensor data, wherein the low-level encoder and the high-level encoder were both trained using a training set comprising sequences of sensor data spanning time durations greater than the time duration associated with each subset of sensor data.

In some examples, the first time duration is greater than about 30 seconds.

In some examples, the time duration spanned by each subset of sensor data is less than about two seconds.

In some examples, the method further includes identifying at least one action to be performed by a user device associated with the one or more sensors based on the prediction of the activity the user was engaged in during the collection of the sequence of sensor data. In some examples, the at least one action comprises: causing information relevant to the activity the user was engaged in to be presented, causing a playlist of media content items to begin being presented, causing a pre-defined scripted set of activities to be executed.

In some examples, the one or more sensors are disposed at different locations on a body of the user, and wherein the different locations comprise: a head of the user, a wrist of the user, a finger of the user, a torso of the user, a foot of the user, and/or a leg of the user.

In some examples, the one or more sensors are embedded into a wearable device.

In some examples, the activity the user was engaged in comprises user motion.

In some examples, the low-level encoder and the high-level encoder execute on different compute platforms.

In some aspects, a system for identifying activities includes: a memory; and one or more processors communicatively coupled to the memory. In some aspects, the one or more processors are configured to: obtain a training set, wherein the training set comprises a plurality of training samples, a training sample of the plurality of training samples comprising: a sequence of sensor data obtained from one or more sensors disposed on a user spanning a first time duration, and a label of an activity the user was engaged in during collection of the sequence of sensor data; train a two-stage neural network to generate, for each training sample in the training set, an output indicating a corresponding label of the activity, wherein the two-stage neural network comprises: a low-level encoder configured to receive a subset of the sequence of sensor data spanning a second time duration as input and generate a low-level output, the second time duration shorter than the first time duration, and a high-level encoder configured to receive a plurality of low-level outputs generated by the low-level encoder on a plurality of subsets of the sequence of sensor data and generate the output indicating the corresponding label of the activity; and provide one or more parameters associated with a trained two-stage neural network to a user device, such that the user device uses the one or more parameters to identify activities based on sensor data.

In some examples, the sequence of sensor data comprises at least one of: accelerometer data, gyroscope data, pressure sensor data, magnetometer data, or ambient light senor data.

In some examples, to train the two-stage network, the one or more processors are further configured to: determine, for a training sample in the training set, an error associated with a predicted activity generated by the high-level encoder relative to the corresponding label of the activity; and update weights for the low-level encoder and the high-level encoder based on the error.

In some aspects, a system for identifying activities includes:: a memory; and one or more processors communicatively coupled to the memory. In some aspects, the one or more processors are configured to: obtain a sequence of sensor data obtained from one or more sensors disposed on a user, the sequence of sensor data spanning a first time duration; partition the sequence of sensor data into a plurality of subsets of sensor data, each subset of sensor data spanning a time duration less than the first time duration; provide each subset of the plurality of subsets of sensor data to a low-level encoder, wherein the low-level encoder generates, for each subset of the plurality of subsets of sensor data, a low-level output such that a plurality of low-level outputs corresponding to the plurality of subsets of sensor data is generated by the low-level encoder; and provide the plurality of low-level outputs to a high-level encoder to generate a prediction of an activity the user was engaged in during collection of the sequence of sensor data, wherein the low-level encoder and the high-level encoder were both trained using a training set comprising sequences of sensor data spanning time durations greater than the time duration associated with each subset of sensor data.

In some examples, the one or more processors are further configured to identify at least one action to be performed by a user device associated with the one or more sensors based on the prediction of the activity the user was engaged in during the collection of the sequence of sensor data. In some examples, the at least one action comprises: causing information relevant to the activity the user was engaged in to be presented, causing a playlist of media content items to begin being presented, causing a pre-defined scripted set of activities to be executed.

In some examples, the one or more sensors are disposed at different locations on a body of the user, and wherein the different locations comprise: a head of the user, a wrist of the user, a finger of the user, a torso of the user, a foot of the user, and/or a leg of the user.

In some examples, the one or more sensors are embedded into a wearable device.

In some examples, the low-level encoder and the high-level encoder execute on different compute platforms.

### BRIEF DESCRIPTION OF THE DRAWINGS

Illustrative embodiments are described in detail below with reference to the following figures.

FIG. 1 is a block diagram of an example system for identifying activities using sensor data according to certain embodiments.

FIG. 2 is a schematic diagram of a two-stage network for identifying activities using sensor data according to certain embodiments.

FIGS. 3A and 3B show example representations of outputs of a low-level encoder according to certain embodiments.

FIG. 4 is a flowchart of an example process for training a two-stage network for identifying activities using sensor data according to certain embodiments.

FIG. 5 is a flowchart of an example process for using a trained two-stage network to identify an activity using sensor data according to certain embodiments.

FIG. 6 is a simplified block diagram of an example of a computing system that may be implemented as part of a mobile device and/or a user device according to certain embodiments.

FIG. 7 is a simplified block diagram of an example of a computing system that may be implemented as part of a server according to certain embodiments.

The figures depict embodiments of the present disclosure for purposes of illustration only. One skilled in the art will readily recognize from the following description that alternative embodiments of the structures and methods illustrated may be employed without departing from the principles, or benefits touted, of this disclosure.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Disclosed herein are techniques for identifying high-level activities a user is engaged in based on collected sequences of sensor data. A high-level activity may be a sequence of low-level motion patterns that, together, comprise a high-level user motion. By way of example, a high-level activity of “make breakfast” may include a sequence of low-level motion patterns, such as “open fridge,” “grab butter,” “walk to drawer,” “grab knife,” “spread butter,” or the like. Examples of high-level motion activities include, but are not limited to, “make breakfast,” “morning routine,” “make and drink coffee,” “get ready to leave the house,” “commute to work,” “bedtime routine,” and the like.

In some implementations, a high-level activity may be identified using a two-stage network. The two-stage network may include a low-level encoder, which receives subsets of a sequence of sensor data as inputs and generates low-level encoder outputs (e.g., each corresponding to a subset of a sequence of sensor data). The low-level encoder outputs may then be provided to a high-level encoder which generates a prediction of a high-level activity represented by the sequence of sensor data. In some implementations, a sequence of sensor data may span a relatively longer duration than each subset of the sequence of sensor data provided to the low-level encoder. By way of example, a sequence of sensor data may span 30 seconds, 60 seconds, 90 seconds, 180 seconds, 240 seconds etc., in other words, a duration of time suitable for performing a high-level activity such as “make breakfast.” Continuing with this example, a subset of the sequence of sensor data provided to the low-level encoder may span 0.5 seconds, 1 second, 1.5 seconds, etc., in other words, a duration of time suitable for performing a low-level motion pattern, such as walking from a kitchen counter to the refrigerator, grabbing an object, etc.

In some implementations, a low-level encoder and a high-level encoder may be trained using a training set that includes training samples, where each training sample includes a sequence of sensor data and a corresponding high-level activity label. In other words, the low-level encoder may be trained using high-level activity labels rather than using a training set that includes low-level motion pattern labels. By training the low-level encoder using high-level activity labels, a smaller training set may be used, because thousands of training samples corresponding to each potential low-level pattern are not needed to train the low-level encoder. Moreover, once trained, the low-level encoder may be re-repurposed for identifying low-level motion patterns.

In some implementations, the sensor data may be collected from one or more sensors. The sensors may be of any suitable type, such as accelerometers, gyroscopes, ambient light sensors, magnetometers, pressure sensors, or the like. In some implementations, two or more sensors may be of the same type or of different types. In some embodiments, sensors may be disposed in or on a wearable device (e.g., a smart watch, a fitness tracker, an item of jewelry, smart glasses, a head-mounted display, or the like) and/or embedded in an item of clothing (e.g., a shirt, pants, a hat, a vest, a belt, etc.).

In some implementations, an identified high-level activity may be utilized to trigger an action. The action may include causing a pre-defined script to execute, causing contextually relevant information to be presented, causing particular media content to be presented, or the like. In one example, responsive to predicting, based on a sequence of sensor data, that the high-level activity is “get ready to leave the house,” a pre-defined script that actuates one or more home automation devices in a user's environment may be executed causing, for example, interior house lights to be switched off, causing a home alarm to be activated, etc. In another example, responsive to predicting, based on a sequence of sensor data, that the high-level activity is “bedtime routine,” a “bedtime” playlist of audio content items may be played.

The methods, systems, apparatuses, and media described herein may be used in conjunction with various technologies, such as an artificial reality system. An artificial reality system, such as a head-mounted display (HMD) or heads-up display (HUD) system, generally includes a display configured to present artificial images that depict objects in a virtual environment. The display may present virtual objects or combine images of real objects with virtual objects, as in virtual reality (VR), augmented reality (AR), or mixed reality (MR) applications. For example, in an AR system, a user may view both displayed images of virtual objects (e.g., computer-generated images (CGIs)) and the surrounding environment by, for example, seeing through transparent display glasses or lenses (often referred to as optical see-through) or viewing displayed images of the surrounding environment captured by a camera (often referred to as video see-through). In some AR systems, the artificial images may be presented to users using an LED-based display subsystem.

In some embodiments, the methods, systems, apparatuses, and media described herein may be implemented in connection with a wearable computer, such as a smart watch, a fitness tracker, a HMD, or the like. For example, such a wearable computer may include one or more light emitters and/or one or more light sensors incorporated into a portion of an enclosure of the wearable computer such that light can be emitted toward a tissue of a wearer of the wearable computer that is proximate to or touching the portion of the enclosure of the wearable computer. Example locations of such a portion of an enclosure of a wearable computer may include a portion configured to be proximate to an ear of the wearer (e.g., proximate to a superior tragus, proximate to a superior auricular, proximate to a posterior auricular, proximate to an inferior auricular, or the like), proximate to a forehead of the wearer, proximate to a wrist of the wearer, proximate to a finger tip of the wearer, proximate to a base of a finger of a wearer, proximate to a toe tip of a wearer, or the like.

In the following description, for the purposes of explanation, specific details are set forth in order to provide a thorough understanding of examples of the disclosure. However, it will be apparent that various examples may be practiced without these specific details. For example, devices, systems, structures, assemblies, methods, and other components may be shown as components in block diagram form in order not to obscure the examples in unnecessary detail. In other instances, well-known devices, processes, systems, structures, and techniques may be shown without necessary detail in order to avoid obscuring the examples. The figures and description are not intended to be restrictive. The terms and expressions that have been employed in this disclosure are used as terms of description and not of limitation, and there is no intention in the use of such terms and expressions of excluding any equivalents of the features shown and described or portions thereof. The word “example” is used herein to mean “serving as an example, instance, or illustration.” Any embodiment or design described herein as “example” is not necessarily to be construed as preferred or advantageous over other embodiments or designs.

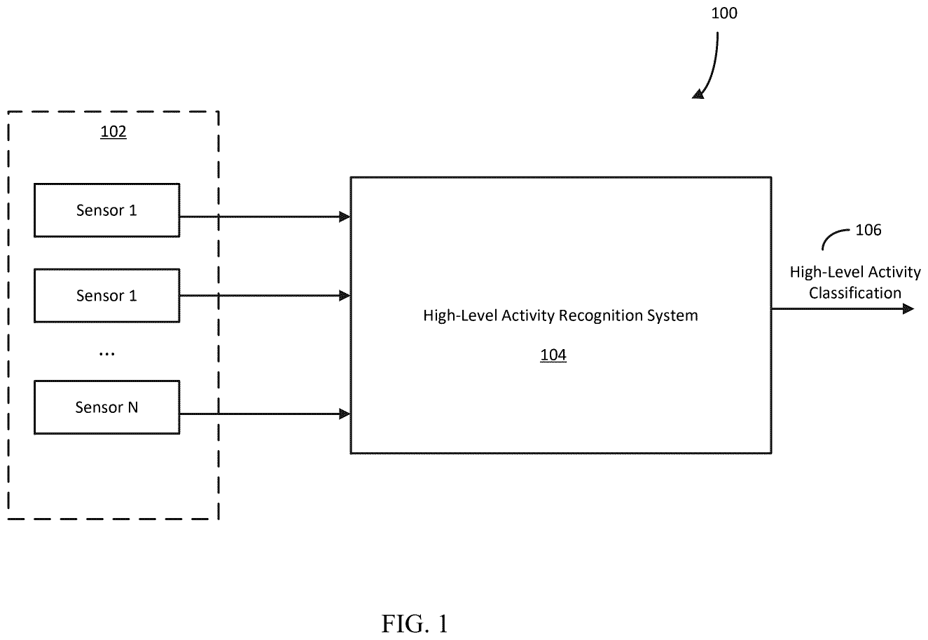

FIG. 1 is a block diagram of an example system 100 for identifying activities using sensor data in accordance with some embodiments. As illustrated, system 100 includes a set of sensors 102. Set of sensors 102 may include any suitable number (e.g., one, two, five, ten, etc.) sensors. In some embodiments, sensors of set of sensors 102 may include any suitable types of sensors, such as one or more accelerometers, one or more gyroscopes, one or more pressure sensors, one or more magnetometers, one or more ambient light detectors, or the like. In some implementations, one or more sensors of set of sensors 102 may be embedded in and/or disposed on a wearable device or object, such as a wrist-worn device (e.g., a smart watch, a fitness tracker, a bracelet, etc.), a finger-worn device (e.g., a ring), a head-mounted device (e.g., glasses, an AR/VR/MR headset, etc.), embedded within a clothing item (e.g., a vest, a shirt, pants, etc.), or the like. In some embodiments, sensors of set of sensors 102 may be proximate to different locations of a wearer's body, such as a head, wrist, finger, torso, feet, legs, or the like.

In some embodiments, data from set of sensors 102 is provided to a high-level activity recognition system 104. In some implementations, high-level activity recognition system 104 may receive, as an input, the data from set of sensors 102, and generate, as an output, a high-level activity classification 106. As described above, high-level activity classification 104 may classify the sensor data as belonging to a particular category of high-level activity. Example categories of high-level activity include: a routine for a particular time of day (e.g., “weekday morning routine,” “weekend morning routine,” “weekday evening routine,” “weekend evening routine,” “bedtime routine,” etc.), performance of a particular task (e.g., “make breakfast,” “wash dishes,” “clean room,” “grocery shopping,” “commute to work,”etc.), or the like.

In some implementations, high-level activity classification 106 may be used to trigger any suitable action. For example, in some implementations, identification of high-level activity classification 106 may trigger performance of a pre-defined (e.g., user-defined) script. As a more particular example, identification of a particular high-level activity classification may trigger a particular home automation sequence to be initiated (e.g., causing lights of a user's home to be switched off responsive to identifying a high-level activity of leaving for work). As another more particular example, identification of high-level activity classification 106 may trigger contextual information to be presented (e.g., causing a weather forecast to be presented responsive to identifying a high-level activity of “get ready to leave the house”). As yet another more particular example, identification of high-level activity classification 106 may trigger particular media content to be presented (e.g., causing a particular bedtime playlist to be played responsive to identifying a high-level activity of “bedtime routine”).

In some implementations, high-level activity recognition system 104 may include a two-stage network. The two-stage network may include a low-level encoder that receives a subset of a stream of sensor data as an input and generates an output. In some implementations, the low-level encoder may generate a sequence of outputs, each corresponding to a subset of the stream of sensor data. For example, in an instance in which the stream of sensor data comprises t seconds (e.g., where t=30 seconds, 60 seconds, 90 seconds, 180 seconds, 240 seconds, etc.), the low-level encoder may receive subsets of the stream of sensor data, where each subset spans a time duration less than t. In one example, the low-level encoder may receive a first subset of the stream of sensor data spanning t₁ seconds (e.g., 0.5 seconds, 1 second, 1.5 seconds, 2 seconds, or the like) and may generate a first low-level output corresponding to the first subset of the stream of sensor data. Continuing with this example, the low-level encoder may receive a second subset of the stream of spanning t₂ seconds (e.g., 0.5 seconds, 1 second, 1.5 seconds, 2 seconds, or the like) and may generate a second low-level output corresponding to the second subset of the stream of sensor data. It should be noted that, in some implementations, t₁ may be the same or may be different from t₂. Moreover, in some implementations, the first subset of the stream of sensor data may overlap the second subset of the stream of sensor data, whereas, in other implementations, the first subset of the stream of sensor data may be non-overlapping.

In some implementations, the output of the low-level encoder may be a tensor or a vector (e.g., having 16 elements, having 32 elements, etc.). Multiple low-level encoder outputs (e.g., corresponding to different time windows of the stream of sensor data) may then be provided as an input to a high-level encoder, which generates, as an output, a classification of a high-level activity associated with the sensor data.

Each of the low-level encoder and the high-level encoder may have any suitable architecture. Example types of architecture include a fully connected network (FCN), a recurrent neural network (RNN), a long short-term memory (LSTM) network, a gated recurrent unit (GRU) network, a one-dimensional convolutional neural network (1-D CNN), and a temporal convolutional network (TCN). In some implementations, the low-level encoder and the high-level encoder may have different architectures. By way of example, the low-level encoder may be an FCN network, and the high-level encoder may be an LSTM network.

In some implementations, the low-level encoder may be configured to receive a subset of stream of sensor data that is on the order of a thousandth to a half of the total stream of sensor data. It should be noted that, in some implementations, a stream of sensor data may include data from multiple sensors (e.g., two sensors, three sensors, five sensors, or the like). The data from the multiple sensors may be time-aligned such that data from multiple sensors span the same time duration and begin and end at substantially the same times. In some implementations, the multiple sensors may be different types of sensors and/or positioned adjacent to different body portions of a wearer. In some implementations, a stream of sensor data may include multiple channels for a single sensor, such as x, y, and z accelerometer data pertaining to an accelerometer.

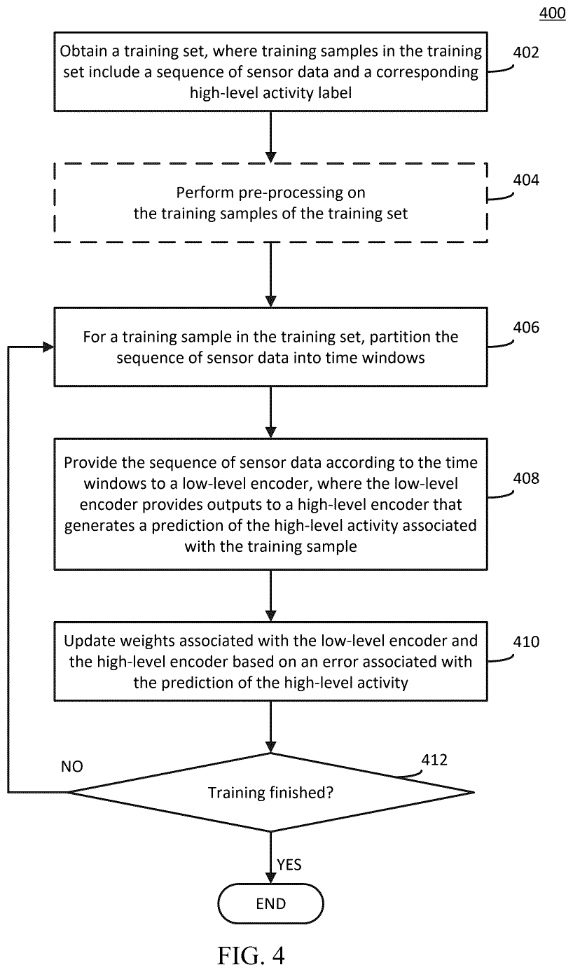

In some implementations, the two-stage network may be trained using a training set that includes training samples (e.g., 100 training samples, 1000 training samples, 10,000 training samples, etc.). A training sample may include a stream of sensor data (e.g., 30 seconds of sensor data, 60 seconds of sensor data, 120 seconds of sensor data, 180 seconds of sensor data, 240 seconds of sensor data, or the like). Each training sample may include a corresponding label of a high-level activity pertaining to the stream of sensor data. The two-stage network may be trained such that weights associated with both the low-level encoder and the high-level encoder are updated based on a loss function associated with prediction of the high-level activity label for a particular stream of sensor data. In other words, the low-level encoder may be trained using only high-level activity labels. More detailed techniques for training such a two-stage network are shown in and described below in connection with FIG. 4.

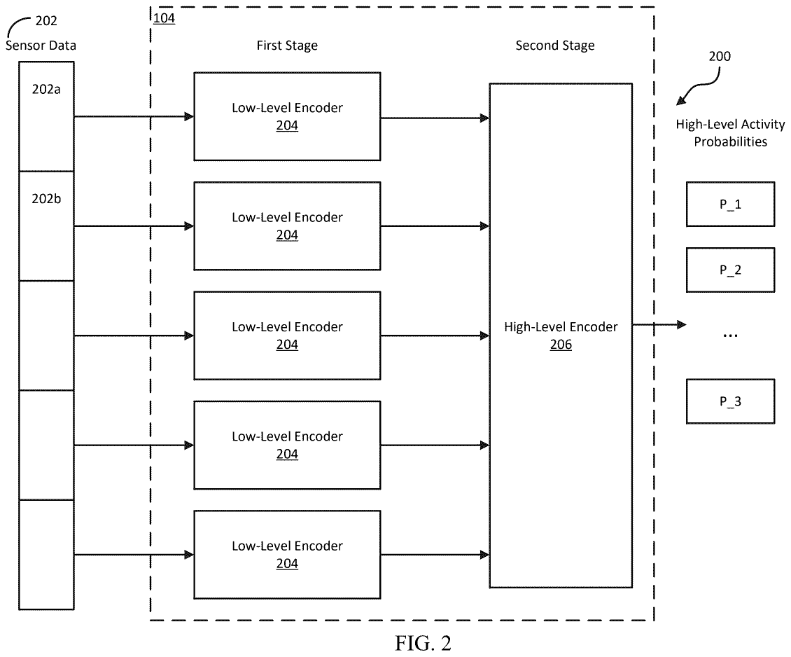

FIG. 2 is a block diagram of an example system 200 for identifying activities using sensor data according to some embodiments. As illustrated, system 200 includes high-level activity recognition system 104, which, as illustrated in FIG. 2, includes a low-level encoder 204 (corresponding to the first stage of a two-stage network) and a high-level encoder 206 (corresponding to a second stage of the two-stage network). Low-level encoder 104 receives a subset of a stream of sensor data 202 as an input. For example, low-level encoder 104 may receive a first subset 202a of stream of sensor data 202. The same low-level encoder 104 may also receive a second subset 202b of stream of sensor data 202. In other words, low-level encoder 104 may be replicated in some implementations such that low-level encoder 104 may receive different subsets of stream of sensor data 202 for analysis. For each subset of stream of sensor data 202, low-level encoder 204 may generate an output which is provided to high-level encoder 206. High-level encoder 206 may then generate an output, which may be used to determine a high-level activity classification. For example, as illustrated in FIG. 2, high-level encoder 206 may generate a vector or tensor, where each element indicates a probability that the sensor data corresponds to a particular high-level activity. Continuing with this example, in some implementations, the sensor data may be classified as associated with a particular high-level activity based on the probabilities generated by high-level encoder 206 (e.g., by selecting the high-level activity associated with the highest probability).

As described above, during training of a two-stage network, both the low-level encoder and the high-level encoder may be trained using a training set that includes labels of high-level activities (e.g., “morning routine,” “commute to work,” “make breakfast,” etc.). Each high-level activity may be composed of a sequence of low-level motion patterns, such as walking, picking up an object, setting an object down, etc. By way of example, a “make breakfast” activity may include a sequence of motion patterns corresponding to “walk,” (e.g., to the fridge), “pick up object,” (e.g., to pick up butter), “walk,” (e.g., to a countertop), “set down object,” (e.g., set down butter), “pick up object,” (e.g., pick up knife), and “spread” (e.g., to spread butter on bread). In some implementations, each motion pattern may have a duration that substantially corresponds to a duration of a subset of the stream of sensor data provided to a low-level encoder. Accordingly, outputs of the low-level encoder may represent a corresponding low-level motion pattern associated with a particular subset of a stream of sensor data. By way of example, given an input subset of a stream of sensor data (e.g., spanning 0.5 seconds, 1 second, 2 seconds, etc.), a low-level encoder may generate an output vector or tensor, where the output vector or tensor indicates a likely low-level motion pattern represented by the subset of the stream of sensor data. It should be noted that, as described above in connection with FIGS. 1 and 2, the low-level encoder is not trained with a training set that includes low-level motion pattern labels, but rather, may be trained with high-level activity labels, corresponding to activities spanning a duration longer than the sensor data provided as input to the low-level encoder.

In some implementations, because a low-level encoder is trained using a training set labeled with high-level activities, context for outputs of the low-level encoder may be imposed by the training set, and the high-level activities selected for representation in the training set. In other words, because the low-level encoder is trained as part of the two-stage network to optimize accurate prediction of high-level activities, choice of high-level activities represented in the training set may reflect in outputs of a trained low-level encoder.

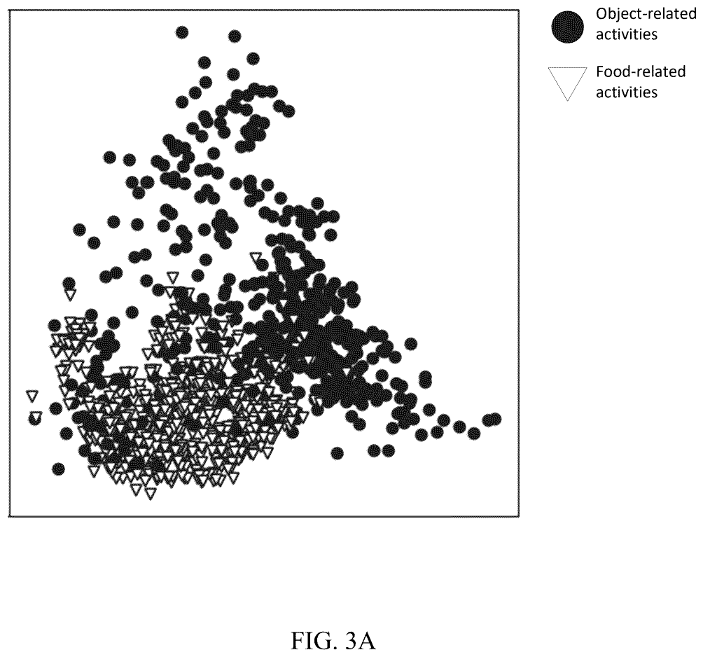

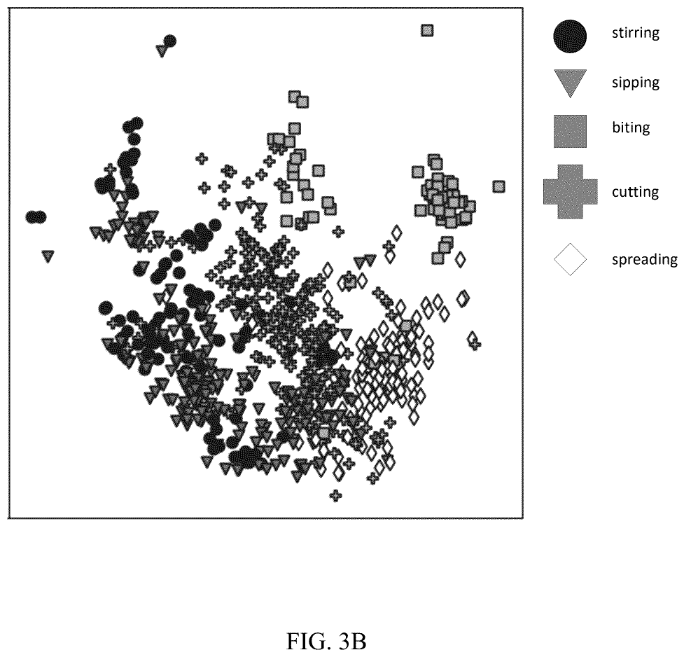

By way of example, FIG. 3A shows a plot of a representation of outputs of a trained low-level encoder. In the example shown in FIG. 3A, a two-stage network, which includes a low-level encoder and a high-level encoder, were trained using a training set that included labeled high-level activities that were either food-related (e.g., “make breakfast,” “morning coffee routine,” etc.) or not. Outputs of the low-level encoder for various subsets of sensor data are shown in FIG. 3A. As illustrated by the triangles in the plot shown in FIG. 3A, subsets of sensor data collected during performance of a food-related high-level activity are classified by the low-level encoder as being more related to each other (e.g., due to being generally located within the cluster of triangles) than subsets of sensor data collected during performance of object-related high-level activities (e.g., due to those subsets of sensor data generally being clustered within the cluster of circles). The low-level encoder outputs included in the cluster of triangles generally correspond to low-level motion patterns such as “sip,” “bite,” “cut,” “stir,” “spread,” and “clean,” i.e., those that may be performed during food-related high-level activities. By contrast, the low-level encoder outputs included in the cluster of circles generally correspond to low-level motion patterns such as “unlock,” “lock,” “open,” and “close,” i.e., those that may be performed during non-food-related high-level activities.

FIG. 3B shows another plot of a representation of outputs of a trained low-level encoder for a training set that included high-level activities of “breakfast time” and “morning coffee.” As illustrated, the outputs of the trained low-level encoder may be clustered into clusters associated with circles, triangles, squares, crosses, and diamonds, corresponding to low-level motion patterns of “stir,” “sip,” “bite,” “cut,” and “spread,” respectively. Note that clusters corresponding to low-level motion patterns of “bite” and “spread” (squares, and diamonds, respectively) are positioned more closely in the plot of FIG. 3B than the clusters corresponding to low-level motion patterns of “stir” and “sip” (circles and triangles, respectively) In particular, because the low-level encoder was trained to produce outputs useful to the high-level encoder for discriminating “breakfast time” (which may involve preparing and eating solid foods that require chewing, cutting, spreading, etc.) from “morning coffee (which may involve preparing and drinking liquid foods that require sipping, stirring, etc.), the low-level encoder has been trained to generate outputs that are more differentiated for low-level motion patterns relevant to distinguishing solid foods versus liquid foods, and generates outputs that are less differentiated for low-level motion patterns involving user body regions utilized for performing the low-level motion patterns. For example, although “bite” and “sip” both involve motion patterns associated with the mouth, the corresponding clusters (e.g., squares and triangles, respectively) are positioned relatively far apart in the plot of FIG. 3B.

It should be noted that representations of a low-level encoder output may be made using any suitable dimensional reduction techniques, such as principal components analysis (PCA), multi-dimensional scaling, or the like. For example, the plots shown in FIGS. 3A and 3B may be generated by applying PCA to low-level encoder outputs such that the outputs are scaled to two dimensions, which may then be plotted in a two-dimensional scatter plot, as shown in FIGS. 3A and 3B.

As illustrated in FIGS. 3A and 3B, once trained, a low-level encoder may generate an output that, for a given subset of a stream of sensor data, generates an output that predicts a low-level motion pattern associated with the subset of the stream of sensor data. A trained low-level encoder may therefore be used to identify low-level motion patterns based on collected sensor data without ever having been trained on labeled low-level motion pattern samples. In other words, a low-level encoder, trained using high-level activity labels, may then be utilized in other contexts for identifying low-level motion patterns. In one example, a trained low-level encoder may be used to identify a stepping motion pattern, a lifting motion pattern, etc., which may be utilized for various purposes (e.g., to initiate a workout application executing on a device, or the like). This may be advantageous, because such a low-level encoder may be trained using relatively fewer training samples than would otherwise be required to train such a low-level encoder. Moreover, the sensor data used to train the low-level encoder may include movements that were naturally performed, as the sensor data was collected during performance of a high-level activity composed of a fluid sequence of low-level motion patterns, rather than a specified low-level motion pattern as part of a calibration routine.

FIG. 4 shows an example of a process 400 for training a two-stage network in accordance with some embodiments. In some implementations, blocks of process 400 may be executed on a server device. In some embodiments, two or more blocks of process 400 may be executed substantially in parallel. In some embodiments, one or more blocks of process 400 may be omitted. In some implementations, blocks of process 400 may be performed in an order other than what is shown in FIG. 4.

Process 400 can begin at 402 by obtaining a training set. In some embodiments, a training sample in the training set includes a sequence of sensor data and a corresponding high-level activity label. In some implementations, a sequence of sensor data may include data from one or more sensors (e.g., one or more accelerometers, gyroscopes, temperature sensors, pressure sensors, or the like). In some implementations, a sequence of sensor data may include sensor data corresponding to multiple axes of motion. In some embodiments, a sequence of sensor data may span any suitable time duration, such as 30 seconds, 60 seconds, 90 seconds, 180 seconds, 240 seconds, or the like. As described above, the high-level activity label may indicate, for the corresponding sequence of sensor data, a particular type of high-level activity the user was engaged in during collection of the sequence of sensor data, where the high-level activity is comprised of a sequence of low-level motion patterns. It should be noted that training samples in the training set may be obtained from the same user, or, in some embodiments, from different users.

In some embodiments, at 404, process 400 can perform pre-processing on the training samples of the training set. For example, in some implementations, process 400 may discard particular training samples as not meeting particular criteria. Examples of training samples that may be discarded include those with sensor data values exceeding particular threshold values, those with null sensor data (e.g., indicating a malfunctioning sensor), or the like. In some implementations, pre-processing may include filtering one or more channels of sensor data. Filtering may include applying a high-pass filter, a low-pass filter, a notch filter, a bandpass filter, and/or any other suitable type of filter or combination of filters.

At 406, process 400 can, for a training samples in the training set, partition the sequence of sensor data into time windows. The sequence of sensor data may be partitioned into any suitable number of time windows (e.g., 30 windows, 60 windows, 90 windows, 180 windows, 270 windows, or the like). In some implementations, a time window may have any suitable duration, e.g., 0.5 seconds, 1 seconds, 1.5 seconds, etc. It should be noted that, the duration of a time window may be a duration suitable to capture a low-level motion pattern (e.g., a step, grabbing an object, lifting an object, unlocking a lock, etc.). In some implementations, shorter time windows may be suitable for capturing relatively jerky low-level motion patterns (e.g., grabbing an object), whereas relatively longer time windows may be suitable for capturing relatively smooth low-level motion patterns (e.g., a series of steps, moving an object from one position to another, etc.). Additionally, it should be noted that the various time windows may have different time durations. For example, a first time window may have a duration of 0.5 seconds, and a second time window may have a duration of 1.5 seconds. Time windows may be overlapping or non-overlapping. In some examples, a first subset of time windows may overlap, and a second subset of time windows may not overlap.

At 408, process 400 can provide the sequence of sensor data, according to the time windows, to a low-level encoder, where the low-level encoder provides outputs to a high-level encoder that generates a prediction of the high-level activity associated with the training samples. For example, in some implementations, as shown in and described above in connection with FIG. 2, process 400 can provide subsets of the sequence of sensor data to the low-level encoder, where each subset of the sequence of sensor data spans a duration of a time window as partitioned at block 406.

In some implementations, portions of a subset of the sequence of sensor data provided to the low-level encoder may be combined (e.g., linearly combined). For example, in some embodiments, multiple channels (e.g., corresponding to different spatial axes) for a particular sensor may be combined. In some embodiments, a mathematical operation, such as a square root, may be applied to a combination of multiple portions of a subset of the sequence of sensor data, for example, to bring a range of the combined sensor data to within an expected range of the low-level encoder.

As described above (e.g., in connection with FIG. 2), multiple low-level encoder outputs, each generated by the low-level encoder responsive to a particular subset of the sequence of the sensor data received as an input by the low-level encoder, are provided to the high-level encoder. The high-level encoder may then generate, based on the multiple low-level encoder outputs, a prediction of the high-level activity corresponding to the training sample.

At 410, process 400 can update weights associated with the low-level encoder and the high-level encoder based on an error associated with the prediction of the high-level activity. It should be noted that any suitable machine learning related techniques may be used to update the weights, such as gradient descent, stochastic gradient descent, or the like. Any suitable learning rate may be used, and, in some embodiments, the learning rate may adapt or change over the course of training. Additionally, it should be noted that, in some implementations, weights may be updated for a batch of training samples.

At 412, process 400 can determine whether training is finished. For example, in some implementations, process 400 can determine whether the prediction error for the training samples has reached a target error. If, at 412, process 400 determines that training is not finished (“no” at 412, process 400 can loop back to 406 and can continue training the two-stage neural network using the training set. Conversely, if, at 412, process 400 determines that training is finished (“yes” at 412), process 400 can end.

In some implementations, parameters (e.g., weights) associated with a trained two-stage network, which may include weights associated with a low-level encoder and weights associated with a high-level encoder, may be utilized at inference time to generate a prediction of a high-level activity associated with a collected sequence of sensor data. In some implementations, the weights may be provided to one or more user devices (e.g., to a wearable device, to a mobile device, etc.) for use at inference time. It should be noted that, in some implementations, inference may be performed using the low-level encoder on a first user device, and inference may be performed using the high-level encoder on a second user device, where the first user device and the second user device are different. In other words, at inference time, the low-level encoder and the high-level encoder may execute on different computing platforms. In such instances, the low-level encoder may transmit low-level encoder outputs to the second user device that executes the high-level encoder such that the high-level encoder can generate the prediction of the high-level activity using the received low-level encoder outputs. In some embodiments, the first user device that executes the low-level encoder may be a wearable device that includes one or more sensors that collect the sequence of sensor data, or an edge device located relatively near the one or more sensors that collect the sequence of sensor data. In some embodiments, the second user device that executes the high-level encoder may be a mobile device (e.g., a mobile phone paired with a wearable device), a tablet computer, a desktop computer, or a remote device, e.g., in the “cloud.”

In some embodiments, a predicted high-level activity corresponding to a collected sequence of sensor data may be used to trigger an action. In some embodiments, the action may include causing a pre-defined script to execute. In some implementations, a pre-defined script may indicate one or more other devices in the user's environment (e.g., other than a user device used at inference time to predict the high-level activity) to perform one or more actions. For example, such other devices may include smart appliances (e.g., a smart thermostat, a door camera, a door lock or opener, smart lights, a home alarm, or the like), a virtual assistant device, or various other types of Internet of Things (IoT) devices. In one example, a pre-defined script may indicate that responsive to a particular type of high-level activity being detected (e.g., “get ready to leave the house”), a particular action is to be performed via one or more other devices in the user's environment (e.g., “activate front door motion camera,” “turn off interior lights,” etc.).

In some embodiments, the action may include causing contextually relevant information to be presented, where the contextually relevant information is relevant to the high-level activity predicted based on the collected sensor data. In some embodiments, types of contextually relevant information may be paired or associated with particular types of high-level activities. For example, “weather information,” and/or “today's calendar items” may be paired with “get ready to leave the house.” In some implementations, the contextually relevant information may be presented on a wearable device associated with one or more sensors that collect the sequence of data (e.g., on a smart watch display, on a lens of smart glasses, displayed as an augmented reality interface in a headset, etc.), on a display of a mobile phone or other mobile device, presented by a virtual assistant device (e.g., as spoken information, within a visual user interface, and/or in any other suitable manner), or the like.

In some embodiments, the action may include causing particular media content to begin being presented. Example types of media content include video content, audio content, a playlist of video content and/or audio content, live-streamed content, a podcast, a slideshow of images, or the like. In one example, responsive to identifying the high-level activity as “get ready for bed,” media content corresponding to a “bedtime playlist” may begin being played. In some implementations, media content may be presented via a wearable computer associated with the one or more sensors from which a sequence of sensor data is collected, via a paired mobile device, and/or via other media playback devices (e.g., speakers, smart televisions, virtual assistant devices, etc.) in the user's environment. In some embodiments, media playback devices may be automatically identified using any suitable discovery techniques.

In some embodiments, the action that is performed may be user-specified or configured. For example, a user may select particular actions to be paired associated with particular types of high-level activities. In instances in which media content is presented responsive to identifying a particular high-level activity, the media content may be identified by the user. For example, the user may curate a playlist of audio content items or identify a particular podcast to be presented responsive to identifying a particular high-level activity. Alternatively, in some implementations, actions may be automatically identified, e.g., based on historical user preferences, preferences of other users, or the like.

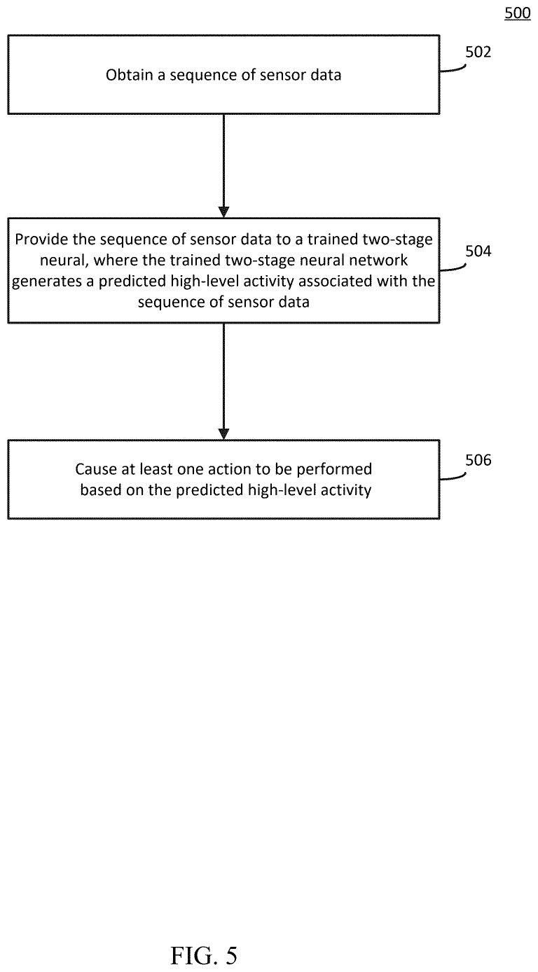

FIG. 5 is a flowchart of an example process 500 for predicting a high-level activity associated with a collected sequence of sensor data and utilizing the predicted high-level activity to perform at least one action in accordance with some embodiments. In some implementations, blocks of process 500 may be performed by the same user device, e.g., that performs inference using a trained low-level encoder and a trained high-level encoder. Alternatively, in some implementations, blocks of process 500 may be performed by two or more different devices, such as a first device that utilizes the trained low-level encoder and a second device that utilizes the trained high-level encoder. In some embodiments, two or more blocks of process 500 may be performed substantially in parallel. In some embodiments, one or more blocks of process 500 may be omitted. In some implementations, blocks of process 500 may be performed in an order other than what is shown in FIG. 5.

Process 500 can begin at 502 by obtaining a sequence of sensor data. In some implementations, the sequence of sensor data may be obtained by one sensor or by multiple (e.g., two, three, five, ten, etc.) sensors. The sensor(s) may be of any suitable type, e.g., one or more accelerometers, one or more gyroscopes, one or more magnetometers, one or more ambient light sensors, one or more pressure sensors, or the like. In some implementations, the sensors may be disposed on or adjacent to a wearable device and/or in or on a wearable clothing item. In some implementations, the sensor data may indicate motion activity of a user from whom the sequence of sensor data was obtained.

At 504, process 500 can provide the sequence of sensor data to a trained two-stage network, where the trained two-stage network generates, as an output, a prediction of the high-level activity the user was engaged in during collection of the sensor data. As described above and as shown in FIG. 2., the two-stage network may include a trained low-level encoder and a trained high-level encoder, where the low-level encoder and the high-level encoder were both trained using a training set comprising sequences of sensor data and labeled high-level activities (e.g., as described above in connection with FIG. 4).

In some implementations, process 500 can provide the sequence of sensor data to the trained-two stage network by providing subsets of the sequence of sensor data to the low-level encoder. For example, each subset may correspond to a relatively short time window (e.g., 0.5 seconds, 1 second, 1.5 seconds, etc.) over which a low-level motion pattern may be performed. In some embodiments, process 500 may partition the sequence of sensor data into multiple subsets of the sequence of sensor data, each spanning a time duration shorter than a time duration spanned by the sequence of sensor data. For example, in an instance in which the sequence of sensor data is 60 seconds long, the subsets of the sequence of sensor data provided to the low-level encoder may be 0.5 seconds, 1 second, 2 seconds, or the like. In some implementations, subsets of the sequence of sensor data may at least partially overlap in time. Low-level encoder outputs, each generated by the low-level encoder responsive to a subset of the sequence of sensor data, may then be provided to the high-level encoder, which generates a final output corresponding to the predicted high-level activity.

At 506, process 500 can cause at least one action to be performed based on the predicted high-level activity. As described above, in some implementations, the at least one action may include causing a pre-defined script to be executed. In some implementations, the pre-defined script may cause one or more devices in the user's environment to execute a routine or perform an action or sequence of actions. For example, the one or more devices may be smart appliances, IoT devices, home automation devices, etc. in the user's environment. As another example, the at least one action may include causing contextually relevant information to be presented based on the identified high-level activity. Examples of contextually relevant information may include a weather forecast, items from a user's calendar (e.g., presented as reminders), or the like. As yet another example, the at least one action may include causing media content to be presented.

In some implementations, process 500 may identify the at least one action by accessing pre-configured user settings that associate the at least one action with the high-level activity identified at block 504. In some embodiments, to cause the at least one action to be performed, process 500 may access one or more applications. For example, to cause a playlist of media content items to be presented, process 500 may access a particular media content presentation application. As another example, to present calendar reminders, process 500 may access a calendar application of a user.

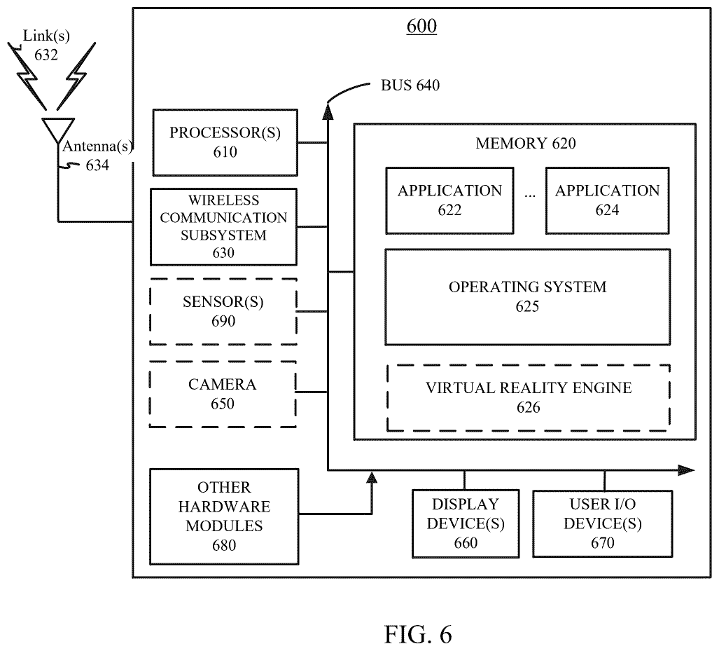

FIG. 6 is a simplified block diagram of an example of a computing system 600 for implementing some of the examples described herein. For example, in some embodiments, computing system may be used to implement a user device (e.g., a mobile device or a wearable computer) that implements the blocks of process 500 shown in and described above in connection with FIG. 5. In the illustrated example, computing system 600 may include one or more processor(s) 610 and a memory 620. Processor(s) 610 may be configured to execute instructions for performing operations at a number of components, and can be, for example, a general-purpose processor or microprocessor suitable for implementation within a portable electronic device. Processor(s) 610 may be communicatively coupled with a plurality of components within computing system 600. To realize this communicative coupling, processor(s) 610 may communicate with the other illustrated components across a bus 640. Bus 640 may be any subsystem adapted to transfer data within computing system 600. Bus 640 may include a plurality of computer buses and additional circuitry to transfer data.

Memory 620 may be coupled to processor(s) 610. In some embodiments, memory 620 may offer both short-term and long-term storage and may be divided into several units. Memory 620 may be volatile, such as static random access memory (SRAM) and/or dynamic random access memory (DRAM) and/or non-volatile, such as read-only memory (ROM), flash memory, and the like. Furthermore, memory 620 may include removable storage devices, such as secure digital (SD) cards. Memory 620 may provide storage of computer-readable instructions, data structures, program modules, and other data for computing system 600. In some embodiments, memory 620 may be distributed into different hardware modules. A set of instructions and/or code might be stored on memory 620. The instructions might take the form of executable code that may be executable by computing system 600, and/or might take the form of source and/or installable code, which, upon compilation and/or installation on computing system 600 (e.g., using any of a variety of generally available compilers, installation programs, compression/decompression utilities, etc.), may take the form of executable code.

In some embodiments, memory 620 may store a plurality of application modules 622 through 624, which may include any number of applications. Examples of applications may include gaming applications, conferencing applications, video playback applications, or other suitable applications. The applications may include a depth sensing function or eye tracking function. Application modules 622-624 may include particular instructions to be executed by processor(s) 610. In some embodiments, certain applications or parts of application modules 622-624 may be executable by other hardware modules 680. In certain embodiments, memory 620 may additionally include secure memory, which may include additional security controls to prevent copying or other unauthorized access to secure information.

In some embodiments, memory 620 may include an operating system 625 loaded therein. Operating system 625 may be operable to initiate the execution of the instructions provided by application modules 622-624 and/or manage other hardware modules 680 as well as interfaces with a wireless communication subsystem 630 which may include one or more wireless transceivers. Operating system 625 may be adapted to perform other operations across the components of computing system 600 including threading, resource management, data storage control and other similar functionality.

Wireless communication subsystem 630 may include, for example, an infrared communication device, a wireless communication device and/or chipset (such as a Bluetooth® device, an IEEE 802.11 device, a Wi-Fi device, a WiMax device, cellular communication facilities, etc.), and/or similar communication interfaces. Computing system 600 may include one or more antennas 634 for wireless communication as part of wireless communication subsystem 630 or as a separate component coupled to any portion of the system. Depending on desired functionality, wireless communication subsystem 630 may include separate transceivers to communicate with base transceiver stations and other wireless devices and access points, which may include communicating with different data networks and/or network types, such as wireless wide-area networks (WWANs), wireless local area networks (WLANs), or wireless personal area networks (WPANs). A WWAN may be, for example, a WiMax (IEEE 802.16) network. A WLAN may be, for example, an IEEE 802.11x network. A WPAN may be, for example, a Bluetooth network, an IEEE 802.6x, or some other types of network. The techniques described herein may also be used for any combination of WWAN, WLAN, and/or WPAN. Wireless communications subsystem 630 may permit data to be exchanged with a network, other computer systems, and/or any other devices described herein. Wireless communication subsystem 630 may include a means for transmitting or receiving data, such as identifiers of HMD devices, position data, a geographic map, a heat map, photos, or videos, using antenna(s) 634 and wireless link(s) 632. Wireless communication subsystem 630, processor(s) 610, and memory 620 may together comprise at least a part of one or more of a means for performing some functions disclosed herein.

Embodiments of computing system 600 may also include one or more sensors 690. Sensor(s) 690 may include, for example, an image sensor, an accelerometer, a pressure sensor, a temperature sensor, a proximity sensor, a magnetometer, a gyroscope, an inertial sensor (e.g., a module that combines an accelerometer and a gyroscope), an ambient light sensor, or any other similar module operable to provide sensory output and/or receive sensory input, such as a depth sensor or a position sensor. For example, in some implementations, sensor(s) 690 may include one or more inertial measurement units (IMUs) and/or one or more position sensors. An IMU may generate calibration data indicating an estimated position of the HMD device relative to an initial position of the HMD device, based on measurement signals received from one or more of the position sensors. A position sensor may generate one or more measurement signals in response to motion of the HMD device. Examples of the position sensors may include, but are not limited to, one or more accelerometers, one or more gyroscopes, one or more magnetometers, another suitable type of sensor that detects motion, a type of sensor used for error correction of the IMU, or some combination thereof. The position sensors may be located external to the IMU, internal to the IMU, or some combination thereof. At least some sensors may use a structured light pattern for sensing.

Computing system 600 may include a display module 660. Display module 660 may be a near-eye display, and may graphically present information, such as images, videos, and various instructions, from computing system 600 to a user. Such information may be derived from one or more application modules 622-624, virtual reality engine 626, one or more other hardware modules 680, a combination thereof, or any other suitable means for resolving graphical content for the user (e.g., by operating system 625). Display module 660 may use liquid crystal display (LCD) technology, light-emitting diode (LED) technology (including, for example, OLED, ILED, μLED, AMOLED, TOLED, etc.), light emitting polymer display (LPD) technology, or some other display technology.

Computing system 600 may include a user input/output module 670. User input/output module 670 may allow a user to send action requests to computing system 600. An action request may be a request to perform a particular action. For example, an action request may be to start or end an application or to perform a particular action within the application. User input/output module 670 may include one or more input devices. Example input devices may include a touchscreen, a touch pad, microphone(s), button(s), dial(s), switch(es), a keyboard, a mouse, a game controller, or any other suitable device for receiving action requests and communicating the received action requests to computing system 600. In some embodiments, user input/output module 670 may provide haptic feedback to the user in accordance with instructions received from computing system 600. For example, the haptic feedback may be provided when an action request is received or has been performed.

Computing system 600 may include a camera 650 that may be used to take photos or videos of a user, for example, for tracking the user's eye position. Camera 650 may also be used to take photos or videos of the environment, for example, for VR, AR, or MR applications. Camera 650 may include, for example, a complementary metal-oxide semiconductor (CMOS) image sensor with a few millions or tens of millions of pixels. In some implementations, camera 650 may include two or more cameras that may be used to capture 3-D images.

In some embodiments, computing system 600 may include a plurality of other hardware modules 680. Each of other hardware modules 680 may be a physical module within computing system 600. While each of other hardware modules 680 may be permanently configured as a structure, some of other hardware modules 680 may be temporarily configured to perform specific functions or temporarily activated. Examples of other hardware modules 680 may include, for example, an audio output and/or input module (e.g., a microphone or speaker), a near field communication (NFC) module, a rechargeable battery, a battery management system, a wired/wireless battery charging system, etc. In some embodiments, one or more functions of other hardware modules 680 may be implemented in software.

In some embodiments, memory 620 of computing system 600 may also store a virtual reality engine 626. Virtual reality engine 626 may execute applications within computing system 700 and receive position information, acceleration information, velocity information, predicted future positions, or some combination thereof of the HMD device from the various sensors. In some embodiments, the information received by virtual reality engine 626 may be used for producing a signal (e.g., display instructions) to display module 660. For example, if the received information indicates that the user has looked to the left, virtual reality engine 626 may generate content for the HMD device that mirrors the user's movement in a virtual environment. Additionally, virtual reality engine 626 may perform an action within an application in response to an action request received from user input/output module 670 and provide feedback to the user. The provided feedback may be visual, audible, or haptic feedback. In some implementations, processor(s) 610 may include one or more GPUs that may execute virtual reality engine 726.

In various implementations, the above-described hardware and modules may be implemented on a single device or on multiple devices that can communicate with one another using wired or wireless connections. For example, in some implementations, some components or modules, such as GPUs, virtual reality engine 626, and applications (e.g., tracking application), may be implemented on a console separate from the head-mounted display device. In some implementations, one console may be connected to or support more than one HMD.

In alternative configurations, different and/or additional components may be included in computing system 600. Similarly, functionality of one or more of the components can be distributed among the components in a manner different from the manner described above. For example, in some embodiments, computing system 600 may be modified to include other system environments, such as an AR system environment and/or an MR environment.

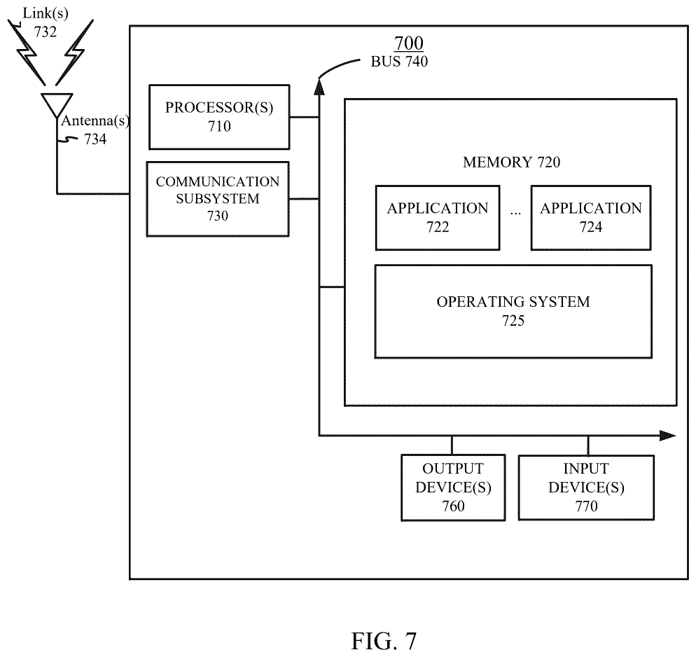

FIG. 7 is a simplified block diagram of an example of a computing system 700 that may be implemented in connection with a server in accordance with some embodiments. For example, computing system 700 may be used to implement a server that generates a trained machine learning model, as described above in connection with FIGS. 2 and 4

In the illustrated example, computing system 700 may include one or more processor(s) 710 and a memory 720. Processor(s) 710 may be configured to execute instructions for performing operations at a number of components, and can be, for example, a general-purpose processor or microprocessor suitable for implementation within a portable electronic device. Processor(s) 710 may be communicatively coupled with a plurality of components within computing system 700. To realize this communicative coupling, processor(s) 710 may communicate with the other illustrated components across a bus 740. Bus 740 may be any subsystem adapted to transfer data within computing system 700. Bus 740 may include a plurality of computer buses and additional circuitry to transfer data. In some embodiments, processor(s) 710 may be configured to perform one or more blocks of process 400, as shown in and described above in connection with FIG. 4, respectively.

Memory 720 may be coupled to processor(s) 710. In some embodiments, memory 720 may offer both short-term and long-term storage and may be divided into several units. Memory 720 may be volatile, such as static random access memory (SRAM) and/or dynamic random access memory (DRAM) and/or non-volatile, such as read-only memory (ROM), flash memory, and the like. Furthermore, memory 720 may include removable storage devices, such as secure digital (SD) cards. Memory 720 may provide storage of computer-readable instructions, data structures, program modules, and other data for computing system 700. In some embodiments, memory 720 may be distributed into different hardware modules. A set of instructions and/or code might be stored on memory 720. The instructions might take the form of executable code that may be executable by computing system 700, and/or might take the form of source and/or installable code, which, upon compilation and/or installation on computing system 700 (e.g., using any of a variety of generally available compilers, installation programs, compression/decompression utilities, etc.), may take the form of executable code.

In some embodiments, memory 720 may store a plurality of application modules 722 through 724, which may include any number of applications. Examples of applications may include gaming applications, conferencing applications, video playback applications, or other suitable applications. Application modules 722-724 may include particular instructions to be executed by processor(s) 710. In some embodiments, certain applications or parts of application modules 722-724 may be executable by other hardware modules 780. In certain embodiments, memory 720 may additionally include secure memory, which may include additional security controls to prevent copying or other unauthorized access to secure information.

In some embodiments, memory 720 may include an operating system 725 loaded therein. Operating system 725 may be operable to initiate the execution of the instructions provided by application modules 722-724 and/or manage other hardware modules 780 as well as interfaces with a wireless communication subsystem 730 which may include one or more wireless transceivers. Operating system 725 may be adapted to perform other operations across the components of computing system 700 including threading, resource management, data storage control and other similar functionality.

Communication subsystem 730 may include, for example, an infrared communication device, a wireless communication device and/or chipset (such as a Bluetooth® device, an IEEE 802.11 device, a Wi-Fi device, a WiMax device, cellular communication facilities, etc.), a wired communication interface, and/or similar communication interfaces. Computing system 700 may include one or more antennas 734 for wireless communication as part of wireless communication subsystem 730 or as a separate component coupled to any portion of the system. Depending on desired functionality, communication subsystem 730 may include separate transceivers to communicate with base transceiver stations and other wireless devices and access points, which may include communicating with different data networks and/or network types, such as wireless wide-area networks (WWANs), wireless local area networks (WLANs), or wireless personal area networks (WPANs). A WWAN may be, for example, a WiMax (IEEE 802.16) network. A WLAN may be, for example, an IEEE 802.11x network. A WPAN may be, for example, a Bluetooth network, an IEEE 802.7x, or some other types of network. The techniques described herein may also be used for any combination of WWAN, WLAN, and/or WPAN. Communications subsystem 730 may permit data to be exchanged with a network, other computer systems, and/or any other devices described herein. Communication subsystem 730 may include a means for transmitting or receiving data, using antenna(s) 734, wireless link(s) 732, or a wired link. Communication subsystem 730, processor(s) 710, and memory 720 may together comprise at least a part of one or more of a means for performing some functions disclosed herein.

In some embodiments, computing system 700 may include one or more output device(s) 760 and/or one or more input device(s) 770. Output device(s) 770 and/or input device(s) 770 may be used to provide output information and/or receive input information.

Embodiments disclosed herein may be used to implement components of an artificial reality system or may be implemented in conjunction with an artificial reality system. Artificial reality is a form of reality that has been adjusted in some manner before presentation to a user, which may include, for example, a virtual reality, an augmented reality, a mixed reality, a hybrid reality, or some combination and/or derivatives thereof. Artificial reality content may include completely generated content or generated content combined with captured (e.g., real-world) content. The artificial reality content may include video, audio, haptic feedback, or some combination thereof, and any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to, for example, create content in an artificial reality and/or are otherwise used in (e.g., perform activities in) an artificial reality. The artificial reality system that provides the artificial reality content may be implemented on various platforms, including an HMD connected to a host computer system, a standalone HMD, a mobile device or computing system, or any other hardware platform capable of providing artificial reality content to one or more viewers.

### Example Embodiments

Embodiment 1: A method for identifying activities, comprising: obtaining a training set, wherein the training set comprises a plurality of training samples, a training sample of the plurality of training samples comprising: a sequence of sensor data obtained from one or more sensors disposed on a user spanning a first time duration, and a label of an activity the user was engaged in during collection of the sequence of sensor data; training a two-stage neural network to generate, for each training sample in the training set, an output indicating a corresponding label of the activity, wherein the two-stage neural network comprises: a low-level encoder configured to receive a subset of the sequence of sensor data spanning a second time duration as input and generate a low-level output, the second time duration shorter than the first time duration, and a high-level encoder configured to receive a plurality of low-level outputs generated by the low-level encoder on a plurality of subsets of the sequence of sensor data and generate the output indicating the corresponding label of the activity; and providing one or more parameters associated with a trained two-stage neural network to a user device, such that the user device uses the one or more parameters to identify activities based on sensor data.

Embodiment 2: the method of embodiment 1, wherein the second time duration is less than about two seconds.

Embodiment 3: the method of any one of embodiments 1 or 2, wherein the first time duration is more than about 30 seconds.

Embodiment 4: the method of any one of embodiments 1-3, wherein the sequence of sensor data comprises at least one of: accelerometer data, gyroscope data, pressure sensor data, magnetometer data, or ambient light senor data.

Embodiment 5: the method of any one of embodiments 1-4, wherein the low-level encoder is a fully connected network, a recurrent neural network, a long short-term (LSTM) network, a gated recurrent unit (GRU) network, a one dimensional convolutional neural network (1-D CNN), or a temporal convolutional network (TCN).

Embodiment 6: the method of any one of embodiments 1-5, wherein the high-level encoder is a fully connected network, a recurrent neural network, a long short-term (LSTM) network, a gated recurrent unit (GRU) network, a one dimensional convolutional neural network (1-D CNN), or a temporal convolutional network (TCN).

Embodiment 7: the method of any one of embodiments 1-6, wherein training the two-stage neural network comprises: partitioning the sequence of sensor data into the plurality of subsets of the sequence of sensor data based on a plurality of time windows; and providing each of the plurality of subsets of the sequence of sensor data to the low-level encoder.

Embodiment 8: the method of any one of embodiments 1-7, wherein each time window of the plurality of time windows is the same.

Embodiment 9: the method of any one of embodiments 1-8, wherein at least two time windows of the plurality of time windows are at least partially overlapping or have different durations.

Embodiment 10: the method of any one of embodiments 1-9, wherein training the two-stage neural network comprises: determining, for a training sample in the training set, an error associated with a predicted activity generated by the high-level encoder relative to the corresponding label of the activity; and updating weights for the low-level encoder and the high-level encoder based on the error.

Embodiment 11: the method of any one of embodiments 1-10, wherein the subset of the sequence of sensor data is filtered by a filter prior to being provided to the low-level encoder, and wherein the filter comprises: a low-pass filter, a high-pass filter, a bandpass filter, or a notch filter.

Embodiment 12: the method of any one of embodiments 1-11, further comprising, prior to providing the plurality of subsets of the sequence of sensor data to the low-level encoder: determining a linear combination of at least a portion of the plurality of subsets of the sequence of sensor data; and applying an arithmetic operation to the linear combination.

Embodiment 13: A method for identifying activities, comprising: obtaining a sequence of sensor data obtained from one or more sensors disposed on a user, the sequence of sensor data spanning a first time duration; partitioning the sequence of sensor data into a plurality of subsets of sensor data, each subset of sensor data spanning a time duration less than the first time duration; providing each subset of the plurality of subsets of sensor data to a low-level encoder, wherein the low-level encoder generates, for each subset of the plurality of subsets of sensor data, a low-level output such that a plurality of low-level outputs corresponding to the plurality of subsets of sensor data is generated by the low-level encoder; and providing the plurality of low-level outputs to a high-level encoder to generate a prediction of an activity the user was engaged in during collection of the sequence of sensor data, wherein the low-level encoder and the high-level encoder were both trained using a training set comprising sequences of sensor data spanning time durations greater than the time duration associated with each subset of sensor data.

Embodiment 14: the method of embodiment 13, wherein the first time duration is greater than about 30 seconds.

Embodiment 15: the method of any one of embodiments 13 or 14, wherein the time duration spanned by each subset of sensor data is less than about two seconds.

Embodiment 16: the method of any one of embodiments 13-15, further comprising identifying at least one action to be performed by a user device associated with the one or more sensors based on the prediction of the activity the user was engaged in during the collection of the sequence of sensor data.

Embodiment 17: the method of embodiment 16, wherein the at least one action comprises: causing information relevant to the activity the user was engaged in to be presented, causing a playlist of media content items to begin being presented, causing a pre-defined scripted set of activities to be executed.

Embodiment 18: the method of any one of embodiments 13-17, wherein the one or more sensors are disposed at different locations on a body of the user, and wherein the different locations comprise: a head of the user, a wrist of the user, a finger of the user, a torso of the user, a foot of the user, and/or a leg of the user.

Embodiment 19: the method of any one of embodiments 13-18, wherein the one or more sensors are embedded into a wearable device.

Embodiment 20: the method of any one of embodiments 13-19, wherein the activity the user was engaged in comprises user motion.

Embodiment 21: the method of any one of embodiments 13-20, wherein the low-level encoder and the high-level encoder execute on different compute platforms.

Embodiment 22: a system for identifying activities, the system comprising: a memory; and one or more processors communicatively coupled with the memory, the one or more processors configured to: obtain a training set, wherein the training set comprises a plurality of training samples, a training sample of the plurality of training samples comprising: a sequence of sensor data obtained from one or more sensors disposed on a user spanning a first time duration, and a label of an activity the user was engaged in during collection of the sequence of sensor data; train a two-stage neural network to generate, for each training sample in the training set, an output indicating a corresponding label of the activity, wherein the two-stage neural network comprises: a low-level encoder configured to receive a subset of the sequence of sensor data spanning a second time duration as input and generate a low-level output, the second time duration shorter than the first time duration, and a high-level encoder configured to receive a plurality of low-level outputs generated by the low-level encoder on a plurality of subsets of the sequence of sensor data and generate the output indicating the corresponding label of the activity; and provide one or more parameters associated with a trained two-stage neural network to a user device, such that the user device uses the one or more parameters to identify activities based on sensor data.

Embodiment 23: the system of embodiment 22, wherein the sequence of sensor data comprises at least one of: accelerometer data, gyroscope data, pressure sensor data, magnetometer data, or ambient light senor data.

Embodiment 24: the system of embodiment 22 or 23, wherein to train the two-stage network, the one or more processors are further configured to: determine, for a training sample in the training set, an error associated with a predicted activity generated by the high-level encoder relative to the corresponding label of the activity; and update weights for the low-level encoder and the high-level encoder based on the error.

Embodiment 25: a system for identifying activities, the system comprising: a memory; and one or more processors communicatively coupled to the memory, the one or more processors configured to: obtain a sequence of sensor data obtained from one or more sensors disposed on a user, the sequence of sensor data spanning a first time duration; partition the sequence of sensor data into a plurality of subsets of sensor data, each subset of sensor data spanning a time duration less than the first time duration; provide each subset of the plurality of subsets of sensor data to a low-level encoder, wherein the low-level encoder generates, for each subset of the plurality of subsets of sensor data, a low-level output such that a plurality of low-level outputs corresponding to the plurality of subsets of sensor data is generated by the low-level encoder; and provide the plurality of low-level outputs to a high-level encoder to generate a prediction of an activity the user was engaged in during collection of the sequence of sensor data, wherein the low-level encoder and the high-level encoder were both trained using a training set comprising sequences of sensor data spanning time durations greater than the time duration associated with each subset of sensor data.

Embodiment 26: the system of embodiment 25, wherein the one or more processors are further configured to identify at least one action to be performed by a user device associated with the one or more sensors based on the prediction of the activity the user was engaged in during the collection of the sequence of sensor data.

Embodiment 27: the system of any one of embodiments 25 or 26, wherein the at least one action comprises: causing information relevant to the activity the user was engaged in to be presented, causing a playlist of media content items to begin being presented, causing a pre-defined scripted set of activities to be executed.

Embodiment 28: the system of any one of embodiments 25-27, wherein the one or more sensors are disposed at different locations on a body of the user, and wherein the different locations comprise: a head of the user, a wrist of the user, a finger of the user, a torso of the user, a foot of the user, and/or a leg of the user.

Embodiment 29: the system of any one of embodiments 25-28, wherein the one or more sensors are embedded into a wearable device.

Embodiment 30: the system of any one of embodiments 25-29, wherein the low-level encoder and the high-level encoder execute on different compute platforms.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

Specific details are given in the description to provide a thorough understanding of the embodiments. However, embodiments may be practiced without these specific details. For example, well-known circuits, processes, systems, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing various embodiments. Various changes may be made in the function and arrangement of elements without departing from the spirit and scope of the present disclosure.

Also, some embodiments were described as processes depicted as flow diagrams or block diagrams. Although each may describe the operations as a sequential process, many of the operations may be performed in parallel or concurrently. In addition, the order of the operations may be rearranged. A process may have additional steps not included in the figure. Furthermore, embodiments of the methods may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It will be apparent to those skilled in the art that substantial variations may be made in accordance with specific requirements. For example, customized or special-purpose hardware might also be used, and/or particular elements might be implemented in hardware, software (including portable software, such as applets, etc.), or both. Further, connection to other computing devices such as network input/output devices may be employed.

With reference to the appended figures, components that can include memory can include non-transitory machine-readable media. The term “machine-readable medium” and “computer-readable medium” may refer to any storage medium that participates in providing data that causes a machine to operate in a specific fashion. In embodiments provided hereinabove, various machine-readable media might be involved in providing instructions/code to processing units and/or other device(s) for execution. Additionally or alternatively, the machine-readable media might be used to store and/or carry such instructions/code. In many implementations, a computer-readable medium is a physical and/or tangible storage medium. Such a medium may take many forms, including, but not limited to, non-volatile media, volatile media, and transmission media. Common forms of computer-readable media include, for example, magnetic and/or optical media such as compact disk (CD) or digital versatile disk (DVD), punch cards, paper tape, any other physical medium with patterns of holes, a RAM, a programmable read-only memory (PROM), an erasable programmable read-only memory (EPROM), a FLASH-EPROM, any other memory chip or cartridge, a carrier wave as described hereinafter, or any other medium from which a computer can read instructions and/or code. A computer program product may include code and/or machine-executable instructions that may represent a procedure, a function, a subprogram, a program, a routine, an application (App), a subroutine, a module, a software package, a class, or any combination of instructions, data structures, or program statements.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that are also expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, etc.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific embodiments have been described, these are not intended to be limiting. Various modifications and equivalents are within the scope of the following claims.

What is claimed is:

1. A method for identifying activities, comprising:
obtaining a training set, wherein the training set comprises a plurality of training samples, a training sample of the plurality of training samples comprising: a sequence of sensor data obtained from one or more sensors disposed on a user spanning a first time duration, and a label of an activity the user was engaged in during collection of the sequence of sensor data;
training a two-stage neural network to generate, for each training sample in the training set, an output indicating a corresponding label of the activity, wherein the two-stage neural network comprises:
a plurality of low-level encoders configured to receive a subset of the sequence of sensor data spanning a second time duration as input and generate a plurality of low-level outputs, the plurality of low-level encoders configured to each receive a different portion of the subset of the sequence of sensor data, the plurality of low-level outputs predicting a low-level motion pattern associated with a respective different portion of the subset of the sequence of sensor data, and the second time duration shorter than the first time duration, and
a high-level encoder configured to receive the plurality of low-level outputs and generate the output indicating the corresponding label of the activity, wherein the respective predicted low-level motion patterns are subsets of high-level motion patterns associated with the activity; and
providing one or more parameters associated with a trained two-stage neural network to a user device, such that the user device uses the one or more parameters to identify activities based on sensor data.

1. The method of claim 1, wherein the second time duration is less than about two seconds.

2. The method of claim 1, wherein the first time duration is more than about 30 seconds.

3. The method of claim 1, wherein the low-level encoders of the plurality of low-level encoders include one or more of a fully connected network, a recurrent neural network, a long short-term (LSTM) network, a gated recurrent unit (GRU) network, a one dimensional convolutional neural network (1-D CNN), or a temporal convolutional network (TCN).

4. The method of claim 1, wherein the high-level encoder is a fully connected network, a recurrent neural network, a long short-term (LSTM) network, a gated recurrent unit (GRU) network, a one dimensional convolutional neural network (1-D CNN), or a temporal convolutional network (TCN).

5. The method of claim 1, wherein training the two-stage neural network comprises:
partitioning the sequence of sensor data into the different portions of the subset of the sequence of sensor data based on a plurality of time windows; and
providing each of the different portions to the plurality of low-level encoders, wherein the plurality of different portions are non-overlapping.

6. The method of claim 5, wherein at least two time windows of the plurality of time windows have different durations.

7. The method of claim 1, wherein training the two-stage neural network comprises:
determining, for a training sample in the training set, an error associated with a predicted activity generated by the high-level encoder relative to the corresponding label of the activity; and
updating weights for the low-level encoder and the high-level encoder based on the error.

8. A method for identifying activities, comprising:
obtaining a sequence of sensor data obtained from one or more sensors disposed on a user, the sequence of sensor data spanning a first time duration;
partitioning the sequence of sensor data into a plurality of subsets of sensor data, each subset of sensor data being different and spanning a time duration less than the first time duration;
providing each subset of the plurality of subsets of sensor data to a low-level encoder of a plurality of low-level encoders, wherein the low-level encoders generate, for the plurality of subsets of sensor data, a plurality of low-level outputs corresponding to the plurality of subsets of sensor data, and the plurality of low-level outputs predict a low-level motion pattern associated with a respective different subset of the sequence of sensor data; and
providing the plurality of low-level outputs to a high-level encoder to generate a prediction of an activity the user was engaged in during collection of the sequence of sensor data, wherein the respective predicted low-level motion patterns are subsets of high-level motion patterns associated with the activity, and wherein the low-level encoder and the high-level encoder were both trained using a training set comprising sequences of sensor data spanning time durations greater than the time duration associated with each subset of sensor data.

9. The method of claim 8, further comprising identifying at least one action to be performed by a user device associated with the one or more sensors based on the prediction of the activity the user was engaged in during the collection of the sequence of sensor data.

10. The method of claim 9, wherein the at least one action comprises:
causing information relevant to the activity the user was engaged in to be presented, causing a playlist of media content items to begin being presented, causing a pre-defined scripted set of activities to be executed.

11. A system for identifying activities, the system comprising:
a memory; and
one or more processors communicatively coupled with the memory, the one or more processors configured to:
obtain a training set, wherein the training set comprises a plurality of training samples, a training sample of the plurality of training samples comprising: a sequence of sensor data obtained from one or more sensors disposed on a user spanning a first time duration, and a label of an activity the user was engaged in during collection of the sequence of sensor data;
train a two-stage neural network to generate, for each training sample in the training set, an output indicating a corresponding label of the activity, wherein the two-stage neural network comprises:
a plurality of low-level encoders configured to receive a subset of the sequence of sensor data spanning a second time duration as input and generate a plurality of low-level outputs, the plurality of low-level encoders configured to each receive a different portion of the subset of the sequence of sensor data, the plurality of low-level outputs predicting a low-level motion pattern associated with a respective different portion of the subset of the sequence of sensor data, and the second time duration shorter than the first time duration, and
a high-level encoder configured to receive the plurality of low-level outputs and generate the output indicating the corresponding label of the activity, wherein the respective predicted low-level motion patterns are subsets of high-level motion patterns associated with the activity; and
provide one or more parameters associated with a trained two-stage neural network to a user device, such that the user device uses the one or more parameters to identify activities based on sensor data.

12. The system of claim 11, wherein the sequence of sensor data comprises at least one of: accelerometer data, gyroscope data, pressure sensor data, magnetometer data, or ambient light senor data.

13. The system of claim 11, wherein to train the two-stage network, the one or more processors are further configured to:
determine, for a training sample in the training set, an error associated with a predicted activity generated by the high-level encoder relative to the corresponding label of the activity; and
update weights for the low-level encoder and the high-level encoder based on the error.

14. A system for identifying activities, the system comprising:
a memory; and
one or more processors communicatively coupled to the memory, the one or more processors configured to:
obtain a sequence of sensor data obtained from one or more sensors disposed on a user, the sequence of sensor data spanning a first time duration;
partition the sequence of sensor data into a plurality of subsets of sensor data, each subset of sensor data being different and spanning a time duration less than the first time duration;
provide each subset of the plurality of subsets of sensor data to a low-level encoder of a plurality of low-level encoders, wherein the low-level encoders generate, for the plurality of subsets of sensor data, a plurality of low-level outputs corresponding to the plurality of subsets of sensor data, and the plurality of low-level outputs predict a low-level motion pattern associated with a respective different subset of the sequence of sensor data; and
provide the plurality of low-level outputs to a high-level encoder to generate a prediction of an activity the user was engaged in during collection of the sequence of sensor data, wherein the respective predicted low-level motion patterns are subsets of high-level motion patterns associated with the activity, and wherein the low-level encoder and the high-level encoder were both trained using a training set comprising sequences of sensor data spanning time durations greater than the time duration associated with each subset of sensor data.

15. The system of claim 14, wherein the one or more processors are further configured to identify at least one action to be performed by a user device associated with the one or more sensors based on the prediction of the activity the user was engaged in during the collection of the sequence of sensor data.

16. The system of claim 15, wherein the at least one action comprises:
causing information relevant to the activity the user was engaged in to be presented, causing a playlist of media content items to begin being presented, causing a pre-defined scripted set of activities to be executed.

17. The system of claim 14, wherein the one or more sensors are disposed at different locations on a body of the user, and wherein the different locations comprise: a head of the user, a wrist of the user, a finger of the user, a torso of the user, a foot of the user, and/or a leg of the user.

18. The system of claim 14, wherein the one or more sensors are embedded into a wearable device.

19. The system of claim 19, wherein the low-level encoder is configured to execute on the wearable device, and the high-level encoder is configured to execute on a different device than the wearable device.
